(19) United States
(12) Patent Application Publication
(10) Pub. No.: US 2024/0182912 A1
(43) Pub. Date: Jun. 6, 2024

(54) TWO-STAGE DYNAMIC CONTROL OVER REDOX STATE IMPROVES CYSTOLIC EXPRESSION OF DISULFIDE CONTAINING PROETEINS IN E. COLI

(71) Applicant: Duke University, Durham, NC (US)

(72) Inventors: Jennifer Noel Hennigan, Durham, NC (US); Michael Lynch, Durham, NC (US); Romel Menacho Melgar, Durham, NC (US)

(21) Appl. No.: 18/556,393

(22) PCT Filed: Apr. 20, 2022
(86) PCT No.: PCT/US22/71816

§ 371 Date: Oct. 20, 2023

Related U.S. Application Data
(60) Provisional application No. 63/177,088, filed on Apr. 20, 2021.

(51) Int. Cl. C12N 15/70; C07K 16/10

(52) U.S. Cl. CPC C12N 15/70; C07K 16/1003; C07K 2317/569

(57) ABSTRACT

Methods and microorganism for expression of a protein requiring at least one disulfide bond for proper folding of the protein are described. The biofermentation methods comprise growth of a microorganism that may conditionally expressing the protein in addition to at least one synthetic metabolic valve designed to regulate of at least one enzyme effective for altering the redox characteristics of the cytosolic environment of the genetically modified microorganism. In a product producing step of the method, an oxidative cytosolic environment is conditionally provided.

### CROSS-REFERENCE

This application is the national phase entry of PCT application, PCT/US22/71816, filed Apr. 20, 2022, which claims the benefit of U.S. Provisional Application No. 63/177,088, filed Apr. 20, 2021, which applications are incorporated herein by reference in their entirety.

### STATEMENT AS TO FEDERALLY SPONSORED RESEARCH

This invention was made with Government support under Federal Grant T32GM008555 awarded by NIH; Grant No: EE0007563 awarded by DOE; and Grant No: R61 AI140485-01 awarded by NIH/NCATS. The Federal Government has certain rights to this invention.

### BACKGROUND

E. coli is a common expression host in both research and industry however, the reducing environment of the cytoplasm creates challenges for soluble expression of proteins with cysteine residues. Approximately 50% of cysteine residues in heterologous proteins form disulfide bonds required for proper folding and activity. These disulfide bonds are reduced in the E. coli cytoplasm resulting in misfolded inactive protein and aggregation in inclusion bodies.

There are two reductase pathways and previous strains have relied on constitutive changes such as reductase deletions (SHuffle, Origami). Knockouts lead to toxicity that slows growth/limits titer. Since knocking out both reducing pathways in E. coli is completely toxic, Shuffle has a mutant peroxidase to maintain some reducing power. Specialized growth conditions are often required for recombinant protein expression, meaning lower temperature and aeration to limit oxidative stress

Previously strains have been engineered to constitutively increase the oxidative potential of E. coli's cytoplasm by deleting key enzymes in reducing pathways including glutathione oxidoreductase (gor) and thioredoxin reductase (trxB). However, constitutive oxidative stress has a toxic effect, slowing growth and limiting conditions for expression. Additionally, overexpression of a disulfide bond isomerase (dsbC) and disulfide bond oxidase (evr1p) have been shown to improve soluble expression of proteins containing disulfide bonds. Specifically, evr1p catalyzes cysteine oxidation and dsbC isomerizes disulfide bonds to improve correct folding when multiple disulfide bonds are present.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter,

By separating the growth and production phase we engineered cytoplasmic redox state with two-stage fermentations where redox state is only altered during the production phase.

In one aspect of the invention methods of expressing a heterologous protein product in a microorganism are fully described. A first step of the method may include providing a genetically modified microorganism. The genetically modified microorganism characterized by means for conditional expression or overexpression of a heterologous protein product and synthetic metabolic valve(s). A second step of the method may include growing the genetically modified microorganism in a growth media comprising a limiting nutrient. In the growth phase, the microorganism maintains a reducing cytosolic environment that is comparable to the reducing cytosolic environment of a microorganism lacking genetic modifications. When a desired biomass of the genetically modified microorganism is maintained, the method further includes transitioning from a growth phase to a stationary and protein producing phase. In this phase, the synthetic metabolic valve(s) resulting in a shift to an oxidative cytosolic environment, and inducing expression or overexpression of the heterologous protein to result in proper folding of the heterologous protein product in an oxidative environment.

In some aspects, the genetically modified microorganism further comprises a chromosomal deletion of a gor, trxB, or gsh A gene. In some aspects the thioredoxin reductase is the trxB gene or enzyme and the glutamate-cysteine ligase is the gshA gene or enzyme. In some aspects, the wherein the genetically modified microorganism is configured to conditionally overexpress a dsbC gene, an evr1 gene, or a combination thereof in the stationary phase. In some aspects, the genetically modified microorganism is an E. coli microorganism. In some as aspects, the gene of the silencing synthetic metabolic valve encode additional enzymes

Other methods, features and/or advantages is, or will become, apparent upon examination of the following figures and detailed description. It is intended that all such additional methods, features, and advantages be included within this description and are protected by the accompanying claims.

### BRIEF DESCRIPTION OF DRAWINGS

The novel features of the invention are set forth with particularity in the claims. A better understanding of the features and advantages of the present invention will be obtained by reference to the following detailed description that sets forth illustrative aspects, in which the principles of the invention are used, and the accompanying drawings of which:

FIG. 1 is a schematic of the thioredoxin and glutaredoxin pathways according to one aspect of the invention.

FIG. 2 is a graph representing synthetic metabolic valves specific for trxB according to one aspect of the invention.

FIG. 3A-B are graphs representing assays of protein oxidation according to one aspect of the invention. FIG. 3A is modified from Hanson, G. T., et. al. The Journal of Biological Chemistry 2004.

FIG. 4 is a graph representing increase of oxidation in response to a trxB synthetic metabolic valve according to one aspect of the invention.

FIG. 5A-B are graphs representing microorganism growth under different conditions according to one aspect of the invention

FIG. 6A-B are graphs representing redox potential and microorganism growth of microorganism strains under different conditions according to one aspect of the invention

FIG. 7 A-B are graphs representing protein expression in microorganism strains under different conditions according to one aspect of the invention.

FIG. 8A-C: 8A-8B are schematics representing expression of a protein in microorganism strains according to one aspect of the invention. 8C is a Western Blot demonstrating expression of a protein in microorganism strains according to one aspect of the invention.

FIG. 9A-C: 9A is schematic representing expression of a protein in microorganism strains according to one aspect of the invention. 9B a SDS-PAGE and 9C a Western Blot demonstrating expression of a protein in microorganism strains according to one aspect of the invention.

FIG. 10A-C: 10A is schematic representing expression of a protein in microorganism strains according to one aspect of the invention. 10B a SDS-PAGE and 10C a Western Blot demonstrating expression of a protein in microorganism strains according to one aspect of the invention.

FIG. 11 is a graph representing protein expression in microorganism strains according to one aspect of the invention.

FIG. 12A-B are graphs representing protein expression and redox as a function of promotor according to one aspect of the invention

FIG. 13A-B are graphs representing protein expression as a function of promotor according to one aspect of the invention as reported in Moreb, E. A. et al ACS Synth Biol 9, 1483-1486 (2020).

FIG. 14 is a graph representing NADPH concentration in microorganism strains according to one aspect of the invention.

FIG. 15 is a graph representing a promotor specific response of a synthetic metabolic valve regulating a udhA gene according to one aspect of the invention.

FIG. 16 is a graph representing a microorganism growth in relationship to a synthetic metabolic valve regulating a udhA gene and aeration growth conditions according to one aspect of the invention.

FIG. 17A-C are graphs representing an overview of engineering the cytoplasmic redox state in E. coli for proper expression of redox sensitive GFP.

FIG. 18A-B are graphs comparing protein expression with a non-redox sensitive GFP.

FIG. 19 is a graph demonstrating redox sensitive GFP activity with hydrogen peroxide addition.

FIG. 20A-C: A and B are schematic overviews of the trxB dynamic control process. FIG. 20C a graph demonstrating trxB expression level in each dynamic control stain.

FIG. 21A-C are graphs demonstrating improved oxidation of redox sensitive GFP with trxB proteolysis.

FIG. 22A-F are graphs representing E. coli growth and expression challenges with respect to media, temperature, and aeration in microfermentation methods.

FIG. 23A-B are graphs representing E. coli growth and expression in microfermentation methods when combined with dynamic control of cytoplasmic reductases and DsbC overexpression.

FIG. 24A-F are graphs representing improved E. coli robustness with respect to temperature and aeration in microfermentation methods.

FIG. 25A-G: FIG. A-F are SDS-PAGE analysis of protein produced from a genetically modified E. coli that includes dynamic control of redox state. FIG. 25F a graph summarizing the SDS-PAGE data.

FIG. 26A-H are SDS-PAGE analysis of protein produced from a genetically modified E. coli that includes dynamic control of redox state.

### DETAILED DESCRIPTION

We now describe improved cytoplasmic expression of disulfide containing recombinant proteins in engineered E. coli with two-stage dynamic control over the redox state, disulfide oxidase and isomerase activities. Recombinant proteins are expressed in a phosphate limited stationary phase coincident with dynamic control over cytoplasmic reducing power. An oxidized environment is created in the cytosol by deleting gor and dynamic reductions trxB and glutamate-cysteine ligase (gshA) levels upon entry into stationary phase. Additionally, increases in correctly folded disulfide bonds are obtained through overexpression of dsbC and evr1p, again upon entry into the stationary phase. Tightly controlled expression and a reducing cytoplasm prior to phosphate depletion enables robust exponential growth and autoinduction of both an oxidative environment and heterologous protein expression upon phosphate depletion. Disulfide containing proteins with improved expression include single chain variable fragments, human hyaluronidase-I, and tissue plasminogen activator which contain 2-17 disulfide bonds. The host strains and plasmids offer a tightly controlled, robust and scalable approach for the expression and purification of disulfide containing proteins.

The engineered strain with dynamic control of redox state has more robust growth compared to SHuffle and more robust expression of the redox sensitive protein, roGFP, in its oxidized form. This strain with dynamic control of redox state was combined with dynamic overexpression of the chaperones disulfide bond isomerase (dsbC) and the sulfhydryl oxidase (Erv1p). Combining these dynamic control features shifts expression of the nanobody VHH72 from the insoluble fraction to predominantly soluble expression. This expression platform permits expression of any nanobodies in the soluble fraction.

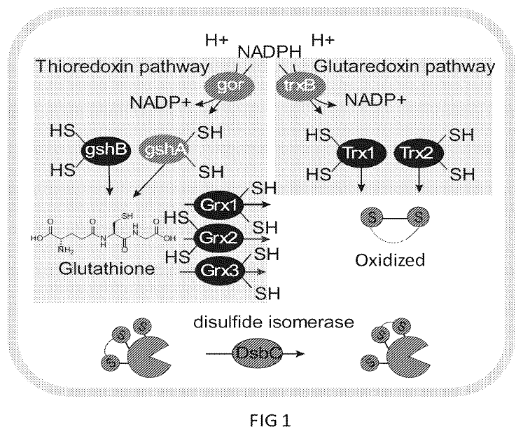

Referring now to FIG. 1, it can be seen that regulation of the gor and trxB genes will have an effect on both a thioredoxin pathway and glutaredoxin pathway of cytosolic redox status. Together with an expression or overexpression of dsbC, an environment amenable to proper folding and disulfibe bond formation in a desired protein product is achieved.

### I. Definitions

Unless otherwise defined, all technical and scientific terms used herein have the same meaning as commonly understood by one of ordinary skill in the art to which this invention pertains. In case of conflict, the present specification, including definitions, will control.

Unless otherwise specified, “a,” “an,” “the,” “one or more of,” and “at least one” are used interchangeably. The singular forms “a”, “an,” and “the” are inclusive of their plural forms.

The recitations of numerical ranges by endpoints include all numbers subsumed within that range (e.g., 0.5 to 5 includes 1, 1.5, 2, 2.75, 3, 3.80, 4, 5, etc.).

The term “about,” when referring to a value or to an amount of mass, weight, time, volume, concentration, or percentage is meant to encompass variations of ±10% from the specified amount. The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

Moreover, the present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid, such as a nonnative promoter driving gene expression. The term “heterologous” is intended to include the term “exogenous” as the latter term is generally used in the art. With reference to the host microorganism's genome prior to the introduction of a heterologous nucleic acid sequence, the nucleic acid sequence that codes for the enzyme is heterologous (whether or not the heterologous nucleic acid sequence is introduced into that genome). As used herein, chromosomal and native and endogenous refer to genetic material of the host microorganism.

As used herein, the term “gene disruption,” or grammatical equivalents thereof (and including “to disrupt enzymatic function,” “disruption of enzymatic function,” and the like), is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduces activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

Bio-production, Micro-fermentation (microfermentation) or Fermentation, as used herein, may be aerobic, microaerobic, or anaerobic.

When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Species and other phylogenic identifications are according to the classification known to a person skilled in the art of microbiology.

Enzymes are listed here within, with reference to a UniProt identification number, which would be well known to one skilled in the art. The UniProt database can be accessed at http://www.UniProt.org/. When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, DCW means dry cell weight, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “OD” means optical density, “OD600” means the optical density measured at a photon wavelength of 600 nm, “kDa” means kilodaltons, “g” means the gravitation constant, “bp” means base pair(s), “kbp” means kilobase pair(s), “% w/v” means weight/volume percent, “% v/v” means volume/volume percent, “IPTG” means isopropyl-μ-D-thiogalactopyranoiside, “aTc” means anhydrotetracycline, “RBS” means ribosome binding site, “rpm” means revolutions per minute, “HPLC” means high performance liquid chromatography, and “GC” means gas chromatography.

### Overview of Invention Aspects

In one aspect of the invention methods of expressing a heterologous protein product in a microorganism are fully described. The method is particularly useful for expression of a protein requiring at least one disulfide bond for proper folding of the protein. A first step of the method may include (a) providing a genetically modified microorganism. The genetically modified microorganism characterized by means for conditional expression or overexpression of a heterologous protein product and synthetic metabolic valve(s). The means for conditional expression of a heterologous protein may include for example a plasmid. However, certainly any means of providing to the microorganism the ability to conditional express a protein heterologous to the microorganism are encompassed by the invention. Referring now specifically to the synthetic metabolic valves, they include a gene expression-silencing synthetic metabolic valve characterized by silencing gene expression of one or more genes encoding one or more enzymes; or an enzymatic degradation synthetic metabolic valve characterized by inducing enzymatic degradation of one or more enzymes, or a combination of valves. In some cases the one or more enzymes of each synthetic metabolic valve(s) are the same or different. The synthetic metabolic valves preferably regulate at least one enzyme effective for altering the redox characteristics of the cytosolic environment of the genetically modified microorganism. A second step of the method may include growing the genetically modified microorganism in a growth media comprising a limiting nutrient. In the growth phase, the microorganism maintains a reducing cytosolic environment that is comparable to the reducing cytosolic environment of a microorganism lacking genetic modifications. When a desired biomass of the genetically modified microorganism is maintained, the method further includes transitioning from a growth phase to a stationary and protein producing phase. Several events may be included in the transition phase, such as stopping the growth of the microorganism, inducing the synthetic metabolic valve(s) resulting in a shift to an oxidative cytosolic environment, and inducing expression or overexpression of the heterologous protein. In this way producing the protein under oxidative conditions so that the necessary disulfide bonds are formed for proper folding of the heterologous protein product in an oxidative environment. The oxidative environment generated in the stationary phased genetically modified microorganism produced by induction of the synthetic metabolic valve(s).

In some aspects, the genetically modified microorganism further comprises a chromosomal deletion of a gor, trxB, or gsh A gene.

In some aspects, the synthetic metabolic valve(s) of the genetically modified microorganism are configured to reduce the activity of a thioredoxin reductase and a glutamate-cysteine ligase by transcriptional silencing of the thioredoxin reductase or glutamate-cysteine ligase gene, reducing the amount of thioredoxin reductase or glutamate-cysteine ligase enzyme by selective proteolysis, or both. In some aspects the thioredoxin reductase is the trxB gene or enzyme and the glutamate-cysteine ligase is the gshA gene or enzyme.

In some aspects, the genetically modified microorganism is configured to conditionally overexpress a thiol:disulfide interchange protein, a thiol oxidase, or a combination thereof in the stationary phase.

In some aspects, the wherein the genetically modified microorganism is configured to conditionally overexpress a dsbC gene, an evr1 gene, or a combination thereof in the stationary phase.

In some aspects, a single genetically modified microorganism comprises a chromosomal deletion of a gor, trxB, or gsh A gene, synthetic metabolic valves configured to conditionally regulate the trxB gene or enzyme and the gshA gene or enzyme, expression or overexpression of a dsbC gene, a evr1 gene, and a heterologous protein. This genetically modified microorganism is useful in methods of producing significant quantities of the heterologous protein that is properly folded and has disulfide bonds.

In some aspects, the heterologous protein is a human protein, an antibody, an antibody fragment, a single chain variable fragment of an antibody, a hyaluronidase-I, a tissue plasminogen activator, a nanobody, or a protein that is approximately 14 kDa in size. It is appreciated that in some aspects nanobodies or antibody fragments may be produced by the dynamic control redox strains according to aspects of the invention. The nanobody or antibody fragment may be about 14 kDa in size. It is appreciated however, that any nanobody or antibody fragment that is between 10 kDa and 25 kDa is encompassed as an aspect of the present invention. Preferably the fragments between 10 kDa and 25 kDa maintains the function of the 14 kDa peptides described in the examples herein as far as functionality.

In some as aspects, microorganism growth is stopped in the transition phrase by phosphate depletion of the growth media.

In some aspects, the genetically modified microorganism is an E. coli microorganism.

In some as aspects, the gene of the silencing synthetic metabolic valve encodes an enzyme selected from the group: enoyl-ACP reductase (fabl), citrate synthase (gitA), soluble transhydrogenase (udhA), glucose-6-phosphate-l-dehydrogenase (zwf), or lipoamide dehydrogenase (lpd), or a combinations of these; or the enzyme of the enzymatic degradation synthetic metabolic valve is selected from the group of: enoyl-ACP reductase (fabI), citrate synthase (gitA), soluble transhydrogenase (udhA), glucose-6-phosphate-l-dehydrogenase (zwf), or lipoamide dehydrogenase (lpd), or combinations of these.

In some cases the synthetic metabolic valve effective for gene-silencing further includes (i) a gene encoding at least one small guide RNA specific for targeting more than one gene; (ii) a Cascade protein complex wherein cas3 is absent or cas3 is modified to form part of the Cascade protein complex but also lacks enzyme activity. In use, then, the (i) gene and the (ii) Cascade protein complex will remain inhibited or suppressed when inorganic phosphate is available to the microorganism, but will be activated in the absence of inorganic phosphate. The synthetic metabolic valve may encode one or more gene for the Cascade protein complex, or otherwise include means that regulate how the Cascade protein complex is assembled in vivo within the genetically modified microorganism.

### Disclosed Aspects are Non-Limiting

While various aspects of the present invention have been shown and described herein, it is emphasized that such aspects are provided by way of example only. Numerous variations, changes and substitutions may be made without departing from the invention herein in its various aspects. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping unless clearly stated otherwise, it is intended that each such grouping provides the basis for and serves to identify various subset aspects, the subset aspects in their broadest scope comprising every subset of such grouping by exclusion of one or more members (or subsets) of the respective stated grouping. Moreover, when any range is described herein, unless clearly stated otherwise, that range includes all values therein and all sub-ranges therein.

Also, and more generally, in accordance with disclosures, discussions, examples and aspects herein, there may be employed conventional molecular biology, cellular biology, microbiology, and recombinant DNA techniques within the skill of the art. Such techniques are explained fully in the literature. See, e.g., Sambrook and Russell, “Molecular Cloning: A Laboratory Manual,” Third Edition 2001 (volumes 1-3), Cold Spring Harbor Laboratory Press, Cold Spring Harbor, N.Y.; Animal Cell Culture, R. I. Freshney, ed., 1986. These published resources are incorporated by reference herein.

The following published resources are incorporated by reference herein for description useful in conjunction with the invention described herein, for example, methods of industrial bio-production of chemical product(s) from sugar sources, and also industrial systems that may be used to achieve such conversion (Biochemical Engineering Fundamentals, 2^{nd} Ed. J. E. Bailey and D. F. Ollis, McGraw Hill, New York, 1986, e.g. Chapter 9, pages 533-657 for biological reactor design; Unit Operations of Chemical Engineering, 5^{th} Ed., W. L. McCabe et al., McGraw Hill, New York 1993, e.g., for process and separation technologies analyses; Equilibrium Staged Separations, P. C. Wankat, Prentice Hall, Englewood Cliffs, NJ USA, 1988, e.g., for separation technologies teachings).

All publications, patents, and patent applications mentioned in this specification are entirely incorporated by reference.

### General Consideration

### I. Carbon Sources

Bio-production media, which is used in the present invention with recombinant microorganisms must contain suitable carbon sources or substrates for both growth and production stages. Suitable substrates may include but are not limited a combination of glucose, sucrose, xylose, mannose, arabinose, oils, carbon dioxide, carbon monoxide, methane, methanol, formaldehyde, or glycerol. It is contemplated that all of the above mentioned carbon substrates and mixtures thereof are suitable in the present invention as a carbon source(s).

### II. Microorganisms

Features as described and claimed herein may be provided in a microorganism selected from the listing herein, or another suitable microorganism, that also comprises one or more natural, introduced, or enhanced product bio-production pathways. Thus, in some embodiments the microorganism(s) comprise an endogenous product production pathway (which may, in some such embodiments, be enhanced), whereas in other embodiments the microorganism does not comprise an endogenous product production pathway.

More particularly, based on the various criteria described herein, suitable microbial hosts for the bio-production of a chemical product generally may include, but are not limited to the organisms described in the Methods Section.

The host microorganism or the source microorganism for any gene or protein described here may be selected from the following list of microorganisms: Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia, Salmonella, Bacillus, Streptomyces, and Pseudomonas. In some aspects the host microorganism is an E. coli microorganism.

### III. Media and Culture Conditions

In addition to an appropriate carbon source, such as selected from one of the herein-disclosed types, bio-production media must contain suitable minerals, salts, cofactors, buffers and other components, known to those skilled in the art, suitable for the growth of the cultures and promotion of chemical product bio-production under the present invention.

Another aspect of the invention regards media and culture conditions that comprise genetically modified microorganisms of the invention and optionally supplements.

Typically cells are grown at a temperature in the range of about 25° C. to about 40° C. in an appropriate medium, as well as up to 70° C. for thermophilic microorganisms. Suitable growth media are well characterized and known in the art. Suitable pH ranges for the bio-production are between pH 2.0 to pH 10.0, where pH 6.0 to pH 8.0 is a typical pH range for the initial condition. However, the actual culture conditions for a particular embodiment are not meant to be limited by these pH ranges. Bio-productions may be performed under aerobic, microaerobic or anaerobic conditions with or without agitation.

### IV. Bio-Production Reactors and Systems

Fermentation systems utilizing methods and/or compositions according to the invention are also within the scope of the invention. Any of the recombinant microorganisms as described and/or referred to herein may be introduced into an industrial bio-production system where the microorganisms convert a carbon source into a product in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to a selected chemical product. Bio-productions may be performed under aerobic, microaerobic, or anaerobic conditions, with or without agitation. Industrial bio-production systems and their operation are well-known to those skilled in the arts of chemical engineering and bioprocess engineering.

The amount of a product produced in a bio-production media generally can be determined using a number of methods known in the art, for example, high performance liquid chromatography (HPLC), gas chromatography (GC), or GC/Mass Spectroscopy (MS).

### V. Genetic Modifications, Nucleotide Sequences, and Amino Acid Sequences

Embodiments of the present invention may result from introduction of an expression vector into a host microorganism, wherein the expression vector contains a nucleic acid sequence coding for an enzyme that is, or is not, normally found in a host microorganism.

The ability to genetically modify a host cell is essential for the production of any genetically modified (recombinant) microorganism. The mode of gene transfer technology may be by electroporation, conjugation, transduction, or natural transformation. A broad range of host conjugative plasmids and drug resistance markers are available. The cloning vectors are tailored to the host organisms based on the nature of antibiotic resistance markers that can function in that host. Also, as disclosed herein, a genetically modified (recombinant) microorganism may comprise modifications other than via plasmid introduction, including modifications to its genomic DNA.

More generally, nucleic acid constructs can be prepared comprising an isolated polynucleotide encoding a polypeptide having enzyme activity operably linked to one or more (several) control sequences that direct the expression of the coding sequence in a microorganism, such as E. coli, under conditions compatible with the control sequences. The isolated polynucleotide may be manipulated to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well established in the art.

The control sequence may be an appropriate promoter sequence, a nucleotide sequence that is recognized by a host cell for expression of a polynucleotide encoding a polypeptide of the present invention. The promoter sequence may contain transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell. The techniques for modifying and utilizing recombinant DNA promoter sequences are well established in the art.

For various embodiments of the invention the genetic manipulations may include a manipulation directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected culture conditions. Genetic manipulation of nucleic acid sequences may increase copy number and/or comprise use of mutants of an enzyme related to product production. Specific methodologies and approaches to achieve such genetic modification are well known to one skilled in the art.

In various embodiments, to function more efficiently, a microorganism may comprise one or more gene deletions. For example, in E. coli, the genes encoding the lactate dehydrogenase (ldhA), phosphate acetyltransferase (pta), pyruvate oxidase (poxB), pyruvate-formate lyase (pflB), methylglyoxal synthase (mgsA), acetate kinase (ackA), alcohol dehydrogenase (adhE), the clpXP protease specificity enhancing factor (sspB), the ATP-dependent Lon protease (lon), the outer membrane protease (ompT), the arcA transcriptional dual regulator (arcA), and the iclR transcriptional regulator (iclR) may be disrupted, including deleted. Such gene disruptions, including deletions, are not meant to be limiting, and may be implemented in various combinations in various embodiments. Gene deletions may be accomplished by numerous strategies well known in the art, as are methods to incorporate foreign DNA into a host chromosome.

In various embodiments, to function more efficiently, a microorganism may comprise one or more synthetic metabolic valves, composed of enzymes targeted for controlled proteolysis, expression silencing or a combination of both controlled proteolysis and expression silencing. For example, one enzyme encoded by one gene or a combination of numerous enzymes encoded by numerous genes in E. coli may be designed as synthetic metabolic valves to alter metabolism and improve product formation. Representative genes in E. coli may include but are not limited to the following: fabI, zwf, gltA, ppc, udhA, lpd, sucD, aceA, pfkA, lon, rpoS, pykA, pykF, tktA or tktB. It is appreciated that it is well known to one skilled in the art how to identify homologues of these genes and or other genes in additional microbial species.

For all nucleic acid and amino acid sequences provided herein, it is appreciated that conservatively modified variants of these sequences are included and are within the scope of the invention in its various embodiments. Functionally equivalent nucleic acid and amino acid sequences (functional variants), which may include conservatively modified variants as well as more extensively varied sequences, which are well within the skill of the person of ordinary skill in the art, and microorganisms comprising these, also are within the scope of various embodiments of the invention, as are methods and systems comprising such sequences and/or microorganisms.

Accordingly, as described in various sections above, some compositions, methods and systems of the present invention comprise providing a genetically modified microorganism that comprises both a production pathway to make a desired product from a central intermediate in combination with synthetic metabolic valves to redistribute flux.

Aspects of the invention also regard provision of multiple genetic modifications to improve microorganism overall effectiveness in converting a selected carbon source into a selected product. Particular combinations are shown, such as in the Examples, to increase specific productivity, volumetric productivity, titer and yield substantially over more basic combinations of genetic modifications.

In addition to the above-described genetic modifications, in various embodiments genetic modifications, including synthetic metabolic valves also are provided to increase the pool and availability of the cofactor NADPH and/or NADH which may be consumed in the production of a product.

### VI. Synthetic Metabolic Valves

Use of synthetic metabolic valves allows for simpler models of metabolic fluxes and physiological demands during a production phase, turning a growing cell into a stationary phase biocatalyst. These synthetic metabolic valves can be used to turn off genes and redirect carbon, electrons, and energy flux to product formation in a multi-stage fermentation process. One or more of the following provides the described synthetic valves: 1) transcriptional gene silencing or repression technologies in combination with 2) inducible and selective enzyme degradation and 3) nutrient limitation to induce a stationary or non-dividing cellular state. SMVs are generalizable to any pathway and microbial host. These synthetic metabolic valves allow for novel rapid metabolic engineering strategies useful for the production of renewable chemicals and fuels and any product that can be produced via whole cell catalysis.

In particular, the invention describes the construction of synthetic metabolic valves comprising one or more or a combination of the following: controlled gene silencing and controlled proteolysis. It is appreciated that one well skilled in the art is aware of several methodologies for gene silencing and controlled proteolysis.

### VI.A Gene Silencing

In particular, the invention describes the use of controlled gene silencing to provide the control over metabolic fluxes in controlled multi-stage fermentation processes. There are several methodologies known in the art for controlled gene silencing, including but not limited to mRNA silencing or RNA interference, silencing via transcriptional repressors and CRISPR interference. Methodologies and mechanisms for RNA interference are taught by Agrawal et al. “RNA Interference: Biology, Mechanism, and Applications” Microbiology and Molecular Biology Reviews, December 2003; 67(4) p 657-685. DOI: 10.1128/MMBR.67.657-685.2003. Methodologies and mechanisms for CRISRPR interference are taught by Qi et al. “Repurposing CRISPR as an RNA-guided platform for sequence-specific control of gene expression” Cell February 2013; 152(5) p 1173-1183. DOI: 10.1016/j.cell.2013.02.022. In addition, methodologies, and mechanisms for CRISRPR interference using the native E. coli CASCADE system are taught by Luo et al. “Repurposing endogenous type I CRISPR-Cas systems for programmable gene repression” NAR. October 2014; DOI: 10.1093. In additional numerous transcriptional repressor systems are well known in the art and can be used to turn off gene expression.

### VI.B Controlled Proteolysis

In particular, the invention describes the use of controlled protein degradation or proteolysis to provide the control over metabolic fluxes in controlled multi-stage fermentation processes. There are several methodologies known in the art for controlled protein degradation, including but not limited to targeted protein cleavage by a specific protease and controlled targeting of proteins for degradation by specific peptide tags. Systems for the use of the E. coli clpXP protease for controlled protein degradation are taught by McGinness et al, “Engineering controllable protein degradation”, Mol Cell. June 2006; 22(5) p 701-707. This methodology relies upon adding a specific C-terminal peptide tag such as a DAS4 (or DAS+4) tag. Proteins with this tag are not degraded by the clpXP protease until the specificity enhancing chaperone sspB is expressed. sspB induces degradation of DAS4 tagged proteins by the clpXP protease. In additional numerous site specific protease systems are well known in the art. Proteins can be engineered to contain a specific target site of a given protease and then cleaved after the controlled expression of the protease. In some embodiments, the cleavage can be expected lead to protein inactivation or degradation. For example Schmidt et al (“ClpS is the recognition component for Escherichia coli substrates of the N-end rule degradation pathway” Molecular Microbiology March 2009. 72(2), 506-517. doi:10.1111), teaches that an N-terminal sequence can be added to a protein of interest in providing clpS dependent clpAP degradation. In addition, this sequence can further be masked by an additional N-terminal sequence, which can be controllable cleaved such as by a ULP hydrolase. This allows for controlled N-rule degradation dependent on hydrolase expression. It is therefore possible to tag proteins for controlled proteolysis either at the N-terminus or C-terminus. The preference of using an N-terminal vs. C-terminal tag will largely depend on whether either tag affects protein function prior to the controlled onset of degradation.

The invention describes the use of controlled protein degradation or proteolysis to provide the control over metabolic fluxes in controlled multi-stage fermentation processes, in E. coli. There are several methodologies known in the art for controlled protein degradation in other microbial hosts, including a wide range of gram-negative as well as gram-positive bacteria, yeast and even archaea. In particular, systems for controlled proteolysis can be transferred from a native microbial host and used in a nonnative host. For example Grilly et al, “A synthetic gene network for tuning protein degradation in Saccharomyces cerevisiae” Molecular Systems Biology 3, Article 127. doi:10.1038, teaches the expression and use of the E. coli clpXP protease in the yeast Saccharomyces cerevisiae. Such approaches can be used to transfer the methodology for synthetic metabolic valves to any genetically tractable host.

### VI.C Synthetic Metabolic Valve Control

In particular the invention describes the use of synthetic metabolic valves to control metabolic fluxes in multi-stage fermentation processes. There are numerous methodologies known in the art to induce expression that can be used at the transition between stages in multi-stage fermentations. These include but are not limited to artificial chemical inducers including: tetracycline, anhydrotetracycline, lactose, IPTG (isopropyl-beta-D-1-thiogalactopyranoside), arabinose, raffinose, tryptophan and numerous others. Systems linking the use of these well-known inducers to the control of gene expression silencing and/or controlled proteolysis can be integrated into genetically modified microbial systems to control the transition between growth and production phases in multi-stage fermentation processes.

In addition, it may be desirable to control the transition between growth and production in multi-stage fermentations by the depletion of one or more limiting nutrients that are consumed during growth. Limiting nutrients can include but are not limited to: phosphate, nitrogen, sulfur, and magnesium. Natural gene expression systems that respond to these nutrient limitations can be used to operably link the control of gene expression silencing and/or controlled proteolysis to the transition between growth and production phases in multi-stage fermentation processes.

### EXAMPLES

For the purposes of promoting an understanding of the principles of the present disclosure, reference will now be made to preferred aspects and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Materials & Methods

Unless otherwise stated, all materials and reagents were of the highest grade possible.

### Example 1: Proteolytic Synthetic Metabolic Regulation of a Thioredoxin Reductase (trxB) Levels

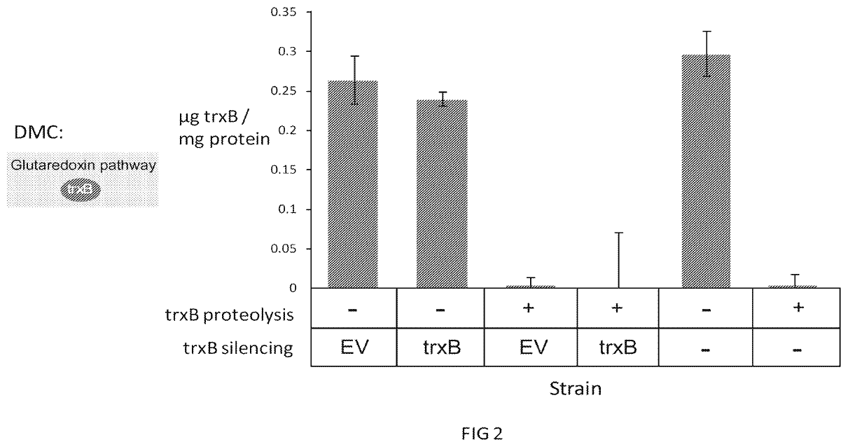

Referring now to FIG. 2, regulation of trxB is affected by the conditional expression of a selective proteolytic valve directed to a trxB enzyme.

### Example 2: An Assay for Protein Oxidation

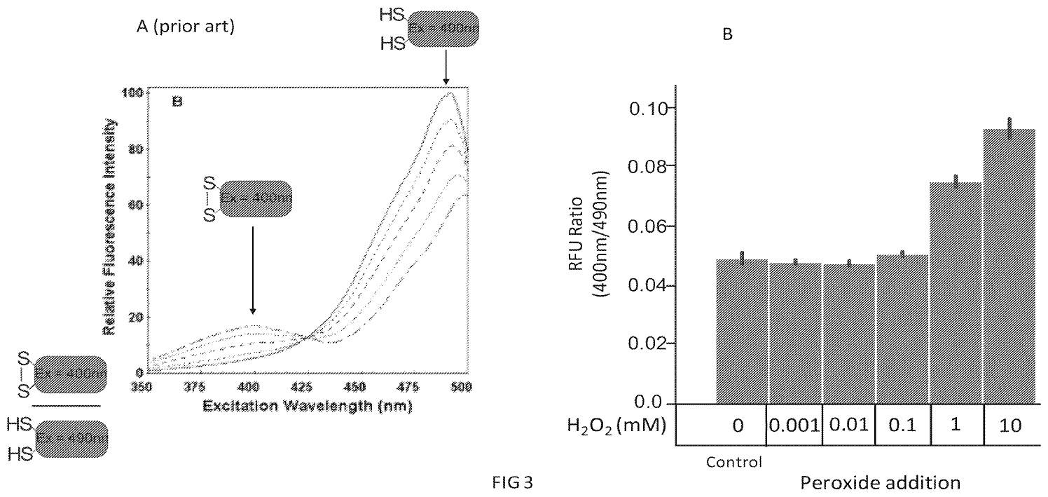

Referring now to FIG. 3A-B, an assay based on relative fluorescence of an exemplary oxidized and reduced protein.

### Example 3: Regulation of trxB Induces an Oxidative Environment

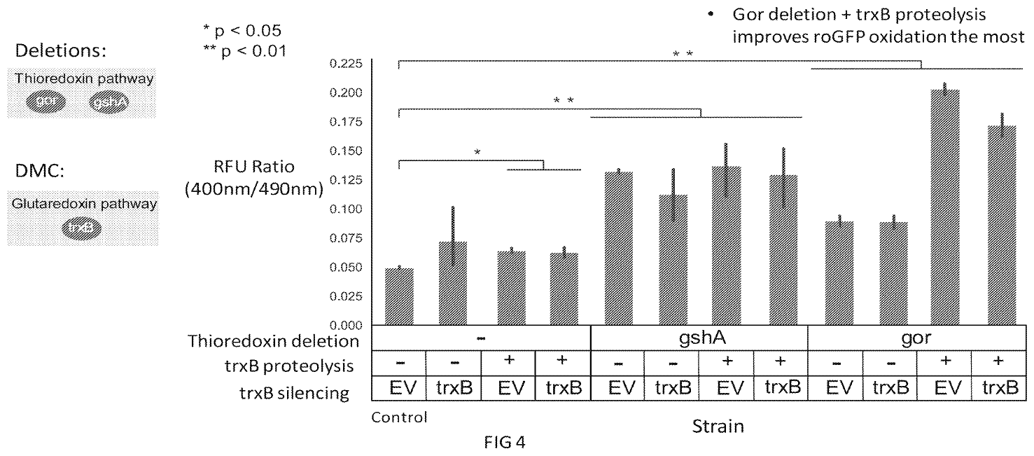

Referring now to FIG. 4, using the assay described above for protein oxidation, the effect of various modifications is described. In particular, the first four bars represent regulation of trxB by either gene silencing or selective proteolysis of the trxB enzyme to increase protein oxidation. The next four bars demonstrate the effectiveness of trxB regulation in the presence of regulation of the gshA enzyme and the final four bars demonstrate the effectiveness of trxB regulation in the presence of regulation of the gor enzyme.

Further, transcriptional silencing of a Lon gene in combination with enhanced proteolysis of trxB enzyme improves expression of proteins that require disulfide bonds for proper folding of the proteins.

### Example 4: Genetically Modified Microorganisms that Conditionally Regulate Cytosolic Redox State are More Robust to Environmental Conditions of Temperature and Aeration

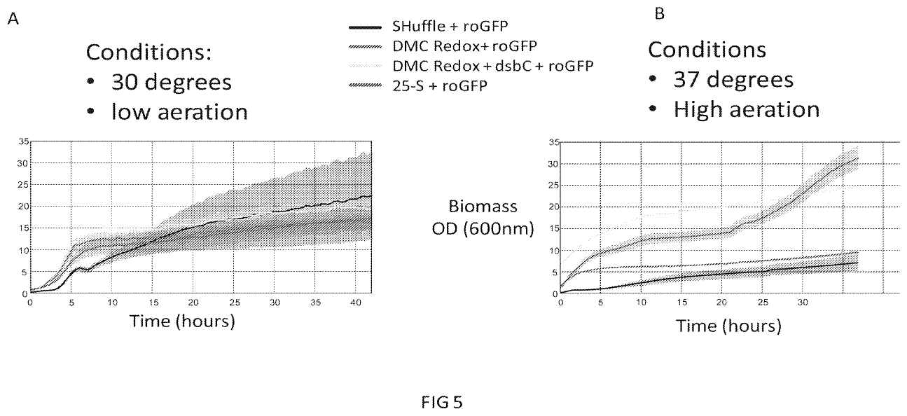

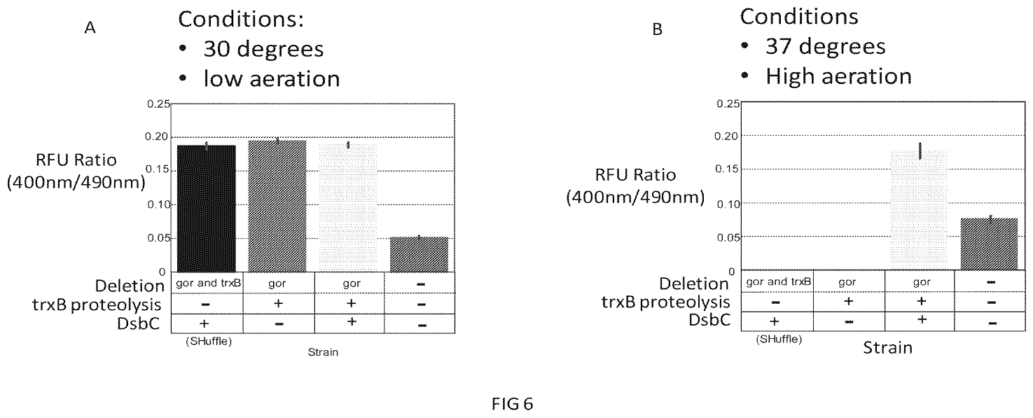

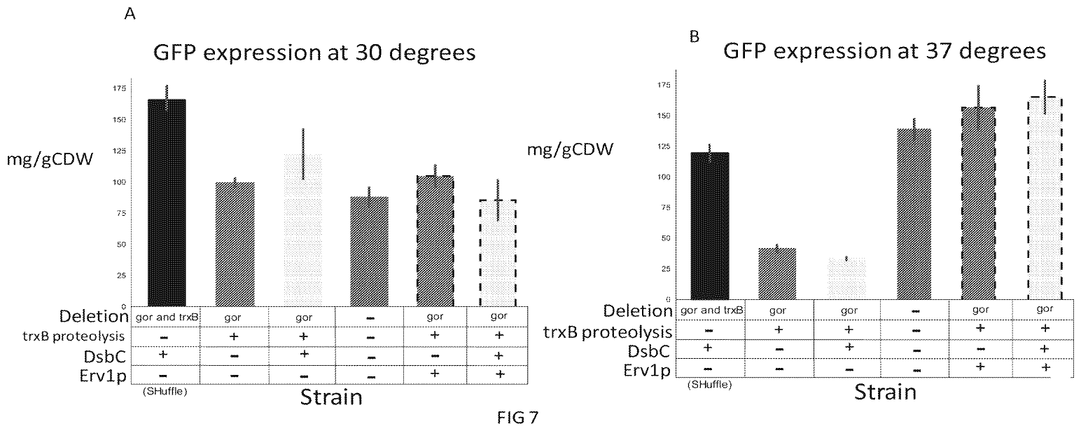

Referring now to FIG. 5-7, microorganisms that conditionally regulate redox conditions have a more robust response to growth under conditions including a temperature of 37° C. and high aeration.

### Example 5: Expression of an Antibody Fragment with Genetically Modified Microorganisms that Conditionally Regulate Cytosolic Redox State

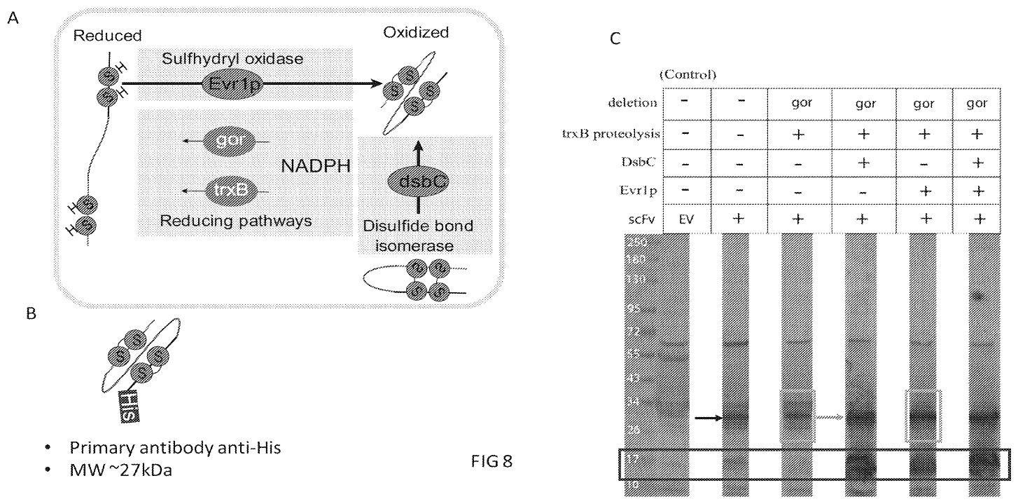

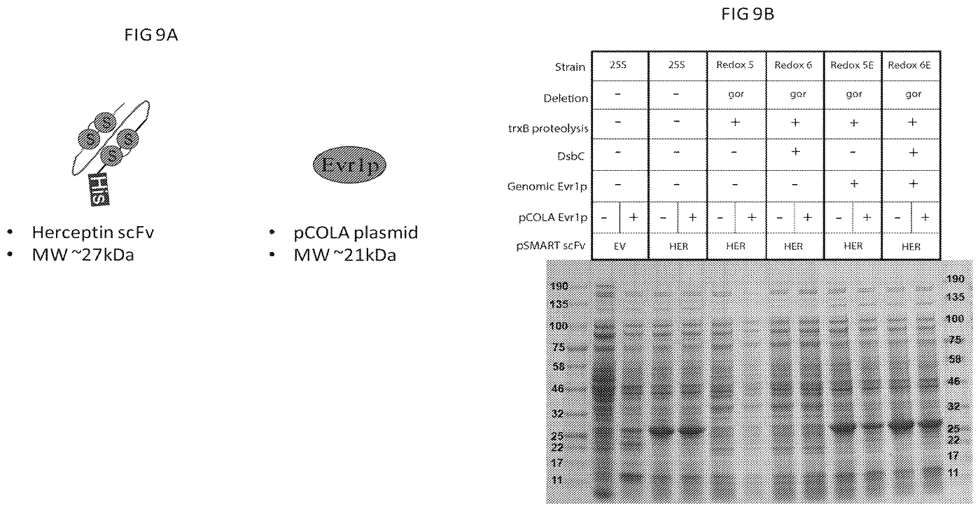

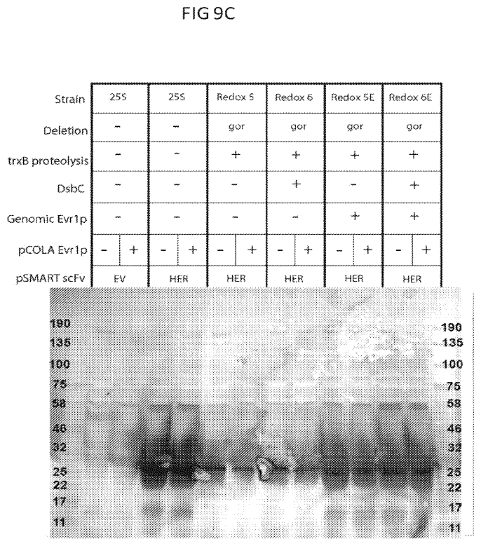

Referring to FIG. 8A-C, a genetically modified microorganism is configured to regulate trxB selective proteolysis with a synthetic metabolic vales, the genetically modified microorganism additionally comprising deletion of the gor gene and expression or overexpression of erv1p or dsbC (FIG. 8A). In this case the antibody fragment is an anti-His scFvs (single chain variable fragment) that is 27 kDa in size (FIG. 8B). As shown in FIG. 8C, the amount of 27 kDa scFvs is improved. Referring to FIG. 9A-C, a Herceptin scFv expresses well with genomic Evr1p. Referring now to FIG. 10A-C, a Herceptin H44-L100 scFv is expressed well in the redox 6E strain with an evr1P pCOLA expression. This construct offers expression advantages with increasing disulfide bonds formed.

### Example 6: Erv1p Rescues Protein Expression with the Microorganism Strains

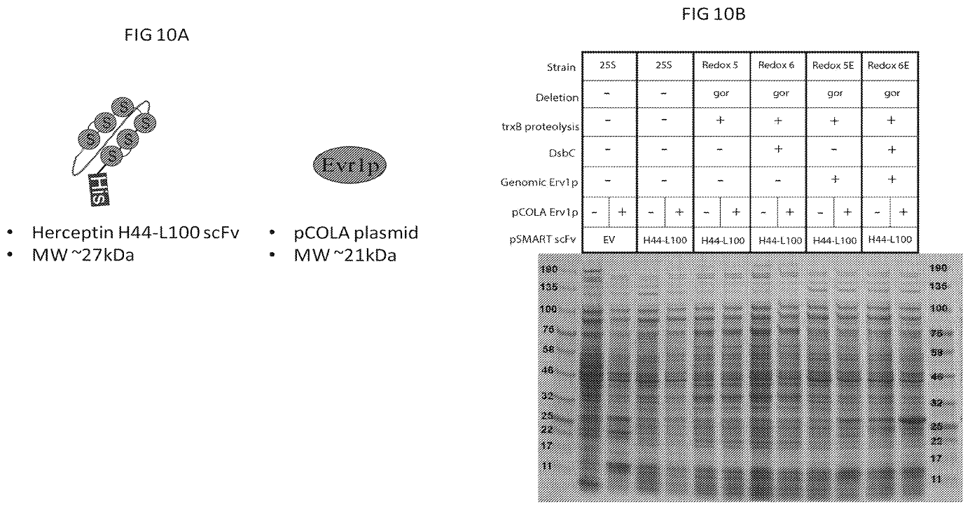

Referring to FIG. 11, protein expression with various microorganism strains indicates expression of an erv1p protein assists in enhancing protein expression in microorganism strains in which trxB is regulated by a synthetic metabolic valve.

### Example 7: Optimization of Promotor for Synthetic Metabolic Valve Expression

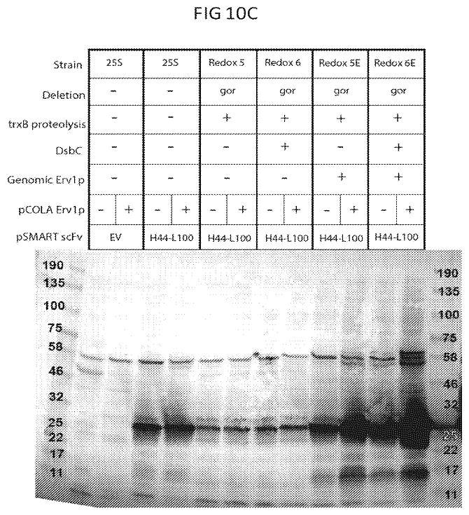

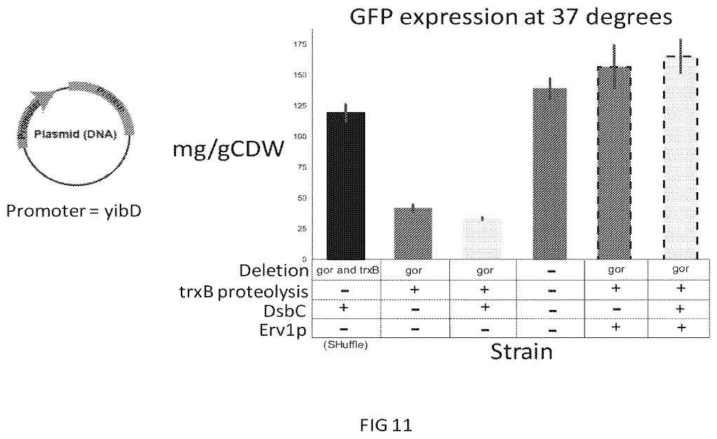

Referring to FIG. 12, limited expression of phoB promotors by redox conditions is shown. In FIG. 12B, a phoB promoter, specifically the phoA promoter is operatively linked to a evr1p thus permitting overexpression of Evr1p so as to rescue recombinant protein expression operatively linked to a set of phoB promoters. Referring to FIG. 13, yibD is shown to be a strong and robust promotor.

### Example 8: A Synthetic Metabolic Valve Directed to udhA Silencing Increases NADPH Concentration

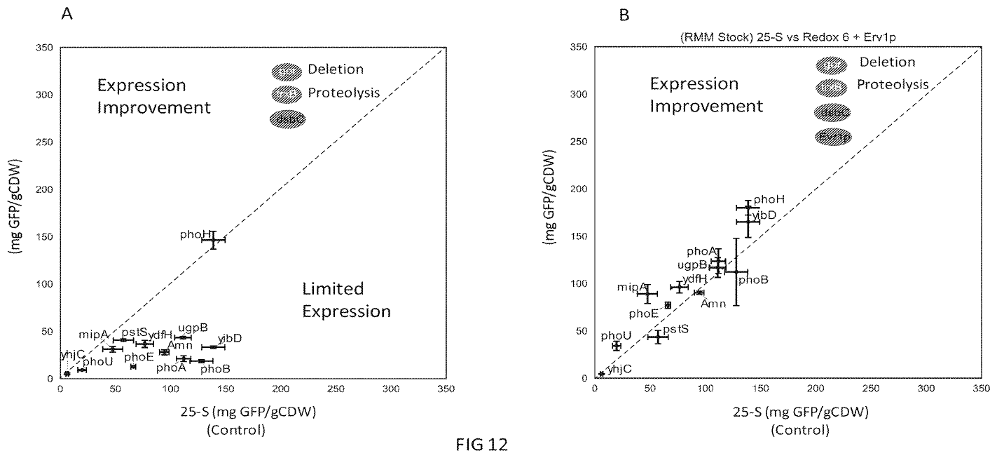

Referring to FIG. 14, a microorganism having a synthetic metabolic valve directed to udhA silencing increases NADPH concentration.

### Example 9: Transcriptional Regulation of a udhA Synthetic Metabolic Valve is Promotor Specific

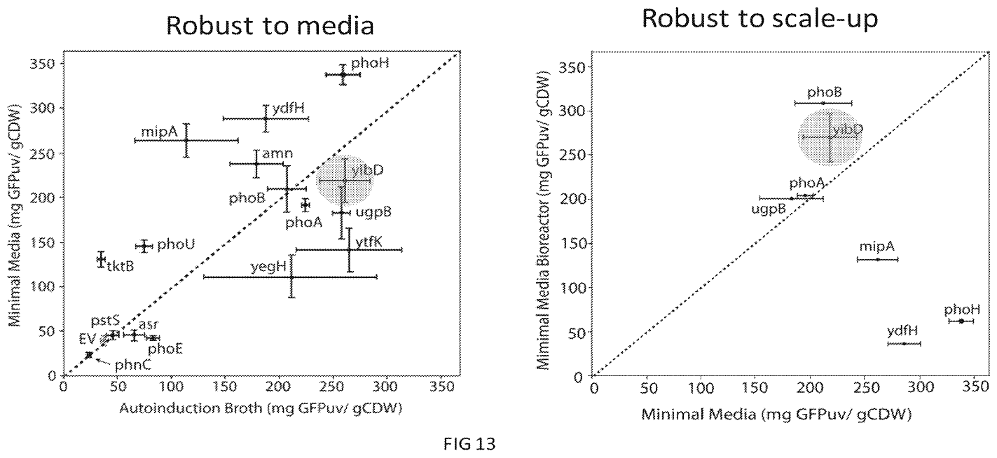

Referring now to FIG. 15, regulation of udhA is dependent on promotor of the synthetic metabolic valve.

### Example 10: Promotors Specific for Aeration and NADPH Sensitivity

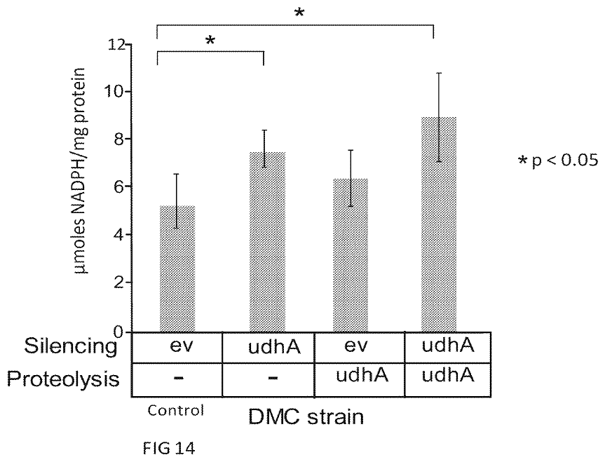

Referring now to FIG. 16, promotors such as yibD, ugpB, ydfH, phoB, and pstS may be used for optimal for production volume and NADPH sensitivity and favorable growth conditions, including increased aeration of the microorganism in the biofermentation process.

### Example 11: Expression of Redox Sensitive GFP

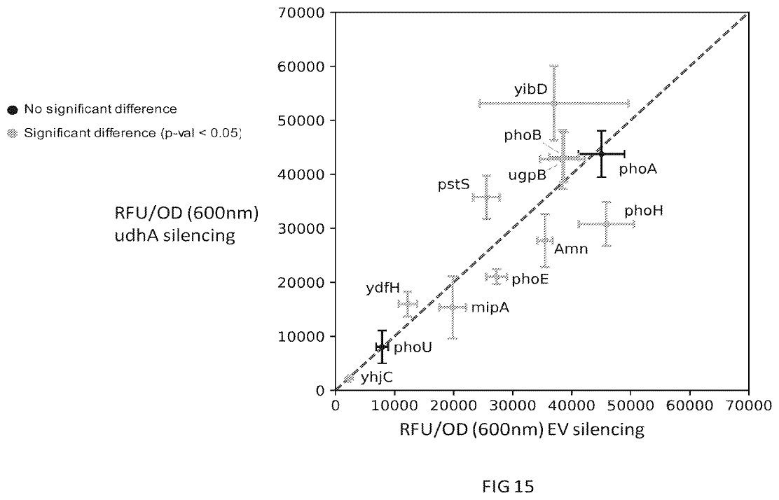

Referring now to FIG. 17, an overview of reducing pathways and engineering cytoplasmic redox state in E. coli with dynamic control is shown. FIG. 17A, Disulfide bonds are reduced to thols in the E. coli cytoplasm by two pathways: the glutaredoxin pathway and the thioredoxin pathway. Key reductases altered to control redox state are shown. The redox sensitive GFP (labeled roGFP) used to assay cytoplasmic redox state is shown. FIG. 17B, Overview of roGFP assay. The excitation spectrum of roGFP changes when it is oxidized vs reduced. An increase in the Emission ratio with excitation at 400 nm over 490 nm indicates greater roGFP oxidation Hydrogen peroxide oxidizes roGFP to confirm the excitation ratio (400 nm/490 nm) increases with increasing oxidation. FIG. 17C, E. coli is engineered for 2-stage fermentations and cytoplasmic oxidation specifically during the production phase to improve disulfide bond formation. The legend in the upper left corner indicates variables. Upon entry into the stationary phase (101), phosphate depletion (102) induces recombinant protein expression of roGFP (103). Simultaneously phosphate depletion triggers controlled reductions in cytoplasmic reductases (104) so disulfides remain oxidized, thereby increasing the excitation ratio (105).

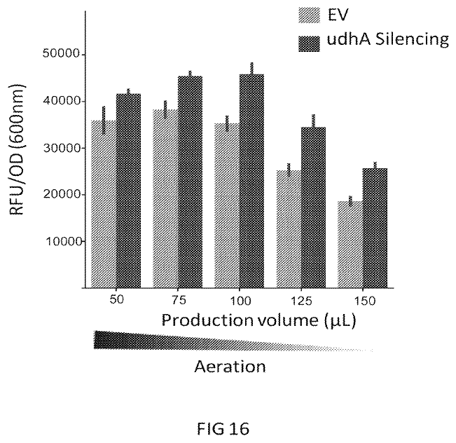

Referring now to FIG. 18, comparison of protein expression in both E. coli strains quantified with a non-redox sensitive GFP. FIG. 18A, GFP expression quantified after micro-fermentations performed at 30° C. in AB autoinduction media with yibDp-GFPuv-pSMART. SHuffle T7 Express is shown on the left and the strain equipped with dynamic control capability (25-S) is shown on the right. FIG. 18B, GFP expression quantified after micro-fermentations performed at 37° C. in AB autoinduction media with the yibDp-GFPuv-pSMART plasmid. Based on non-redox sensitive GFP expression, the strains have different baseline protein expression levels.

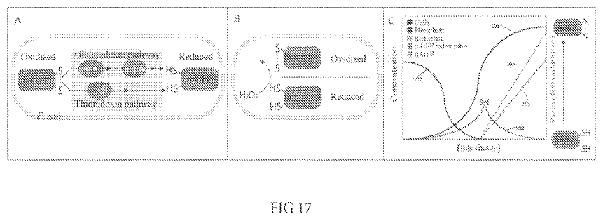

Referring now to FIG. 19, validation of redox sensitive roGFP activity with hydrogen peroxide addition is shown. The excitation ratio is measured after the addition of 0-10 mM hydrogen peroxide to the E. coli control strain (25-S). The micro-fermentations were performed at 37° C. in AB autoinduction media with the yibDp-roGFP-pSMARTplasmid. roGFP is expressed in the control strain without redox control 25-S. Increasing concentrations of hydrogen peroxide to the strain increases roGFP oxidation to indicate cytoplasmic redox state.

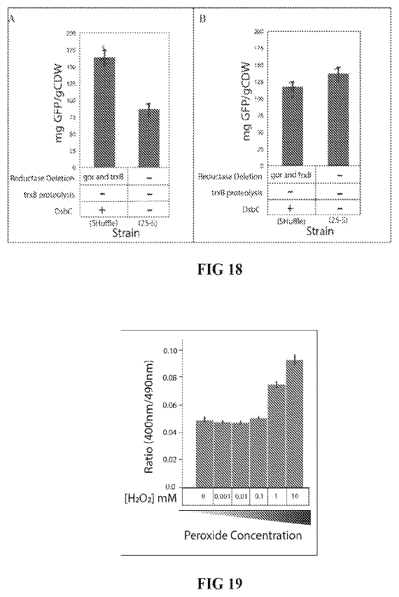

Referring now to FIG. 20, an overview and validation of trxB dynamic control is shown. The trxB was dynamically reduced with two methods: proteolysis and silencing. FIG. 20A, for proteolysis a C-terminal DAS+4 tag was introduced onto trxB. The sspB adapter was controlled by a phoB promoter. Therefore, under phosphate depletion conditions sspB is expressed and joins the trxB DAS4+ tag to the ClpXP protease for controlled proteolysis. FIG. 20B, for silencing a guide RNA (gRNA) specific to the trxB gene was introduced on a plasmid and under the control of a phoB promoter. The subunits of the Cascade complex are also under phoB. Therefore, during the phosphate depleted stationary phase the gRNA was expressed and associated with the Cascade complex to block trxB transcription. FIG. 20C, the trxB expression level in each dynamic control strain is compared to the control strain without proteolysis or silencing. Since the silencing guide was introduced on a plasmid, EV indicates empty vector plasmid and “-” indicates a control without the silencing plasmid. trxB proteolysis significantly decreases the trxB expression level in the strain compared to the control strain (25-S) without trxB dynamic control. trxB silencing did not have a significant impact on trxB expression level in the strain. The empty vector silencing plasmid was not essential for reductions in trxB expression with proteolysis.

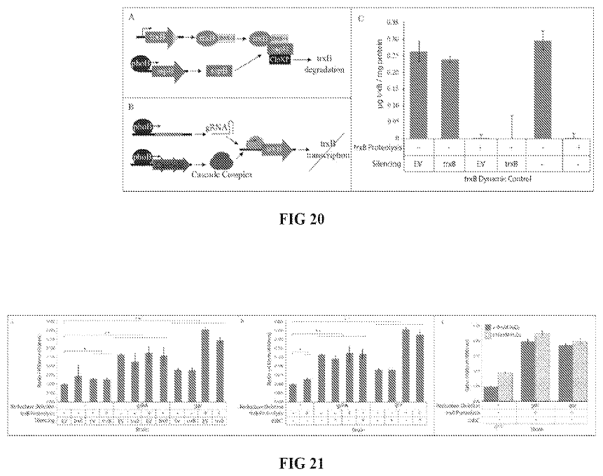

Referring now to FIG. 21, oxidation of roGFP improves with trxB proteolysis is described. In FIG. 21, the roGFP excitation ratio was used to assess cytoplasmic oxidation in each of the E. coli strains with reductase control. Relative to the control strain without reductase control (25-S, leftmost bar). Both trxB dynamic control and glutaredoxin pathway deletions led to significant increases in the excitation ratio with p-values indicated as *p<0.05 and ** p<0.01. FIG. 21B demonstrates incorporating dynamic overexpression of the disulfide bond isomerase (dsbC) in the cytoplasm of these strains resulted in similar levels of cytoplasmic oxidation relative to the control strain. All of these strains had an empty vector silencing plasmid. In FIG. 21C, hydrogen peroxide was added to three strains (patterned bar) to determine if all of the roGFP was oxidized based on comparison of the excitation ratio without hydrogen peroxide addition (solid bar). From left to right, the strains are the control (25-S), the reductase control strain that had the highest cytoplasmic oxidation (Agor; trxB-DAS4+), and strain with combined reductase control and dynamic overexpression of dsbC (Agor; trxB-DAS4+; dsbC). Single modifications of: trxB proteolysis, AgshA, and Agor significantly improved cytoplasmic oxidation over the control strain without reducates control. trxB silencing did not have a significant impact on cytoplasmic oxidation. Combining Agor with trxB proteolysis resulted in the greatest improvement in cytoplasmic oxidation. Adding dynamic overexpression of cytoplasmic dsbC did not hinder cytoplasmic oxidation with the reductase modifications observed in the previous panel. Adding 10 mM hydrogen peroxide to the control strain significantly increases cytoplasmic oxidation of roGFP based on the excitation ratio. The redox excitation of the dynamic control strains are resistant to hydrogen peroxide addition. This indicated that roGFP is predominantly oxidized in these strains before hydrogen peroxide addition.

### Example 12: SHuffle Systems and Dynamic Control Redox Strains

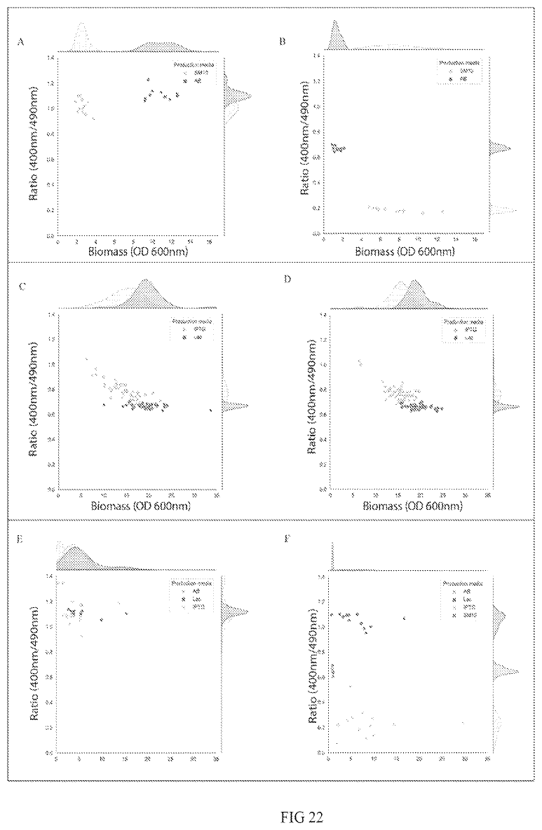

SHuffle E. coli has growth and expression challenges with respect to media, temperature, and aeration in micro-fermentations. Referring now to FIG. 22A, as a control SHuffle E. coli with an EV-pSMART plasmid is grown at 37° C. with high aeration (300 rpm shaking speed) in two types of media for low phosphate induction: SM10 minimal media (light) and AB autoinduction media (dark). Biomass levels and the excitation ratio are reported for each. In FIG. 22B, SHuffle E. coli with yibDp-roGFP-pSMART expression plasmid is grown in the same conditions as FIG. 22A to compare the production OD and the excitation ratio. In FIG. 22C, SHuffle E. coli with an EV-pETM6 plasmid is grown at 30° C. with high aeration (300 rpm shaking speed) in two types of media for T7 induction: LB with IPTG addition (light) and Studier's lac autoinduction media (dark). In FIG. 22D, SHuffle E. coli with the T7-roGFP-pETM6 expression plasmid is grown in the same media and expression conditions as FIG. 22C for comparison. In FIG. 22E, SHuffle E. coli with either EV-pETM6 and EV-pSMART are grown at 30° C. with low aeration (150 rpm shaking speed) in their appropriate induction media indicated by color in the legend. In FIG. 22F, SHuffle E. coli with either T7-roGFP-pETM6 or yibDp-roGFP-pSMART plasmid is grown with the same expression conditions as FIG. 22E to compare production OD and roGFP induction based on the excitation ratio.

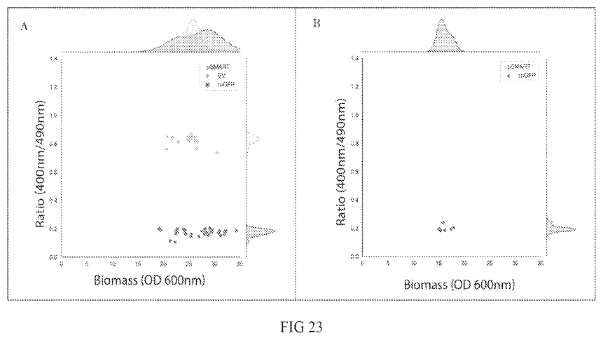

As shown in FIG. 22A, SHuffle E. coli empty vector micro-fermentations with phosphate depletion media at 37° C. with high aeration resulted in low biomass levels in SM10 minimal media and AB autoinduction media. With dynamic control strains Biomass levels >200D600 nm are routinely observed in AB media micro fermentations performed under the same temperature and aeration conditions (see FIG. 23). Due to background emission, excitation ratios close to 1 indicate no expression of roGFP (anticipated with an empty vector). With respect to FIG. 22B, SHuffle E. coli micro-fermentations with yibDp-roGFP at 37° C. with high aeration result in low biomass levels with AB media and lack of roGFP induction based on the excitation ratio. The lack of roGFP induction is likely linked to the limited growth since phosphate is not depleted to trigger autoinduction induction. Interestingly, with yibDp-roGFP in SM10 media SHuffle E. coli had improved growth over the empty vector control in the previous panel and induced it with oxidative cytoplasm. However, based on the range of OD600 nm values (4-140D600 nm) the growth is not robust. Combined with the empty vector data from the previous panel both strain growth and expression of roGFP is not robust at 37° C. with high aeration. With respect to FIG. 22C, final biomass levels improved in SHuffle E. coli micro-fermentations with EV-pETM6 at 30° C. with high aeration in LB media with IPTG induction and Studier's lac autoinduction media. However, biomass levels still had a wide range from 5-20 for LB and 10-35 for Studier's lac indicating a lack of growth robustness. In FIG. 22D, despite the improved biomass levels at 30° C. with high aeration, roGFP did not induce in either media based on a similar excitation ratio as the empty vector control. Combined, there is a lack of growth and expression robustness in SHuffle E. coli. In FIG. 22E, under low temperature (30° C.) and low aeration conditions SHuffle E. coli with empty vectors (pSMART for phosphate depletion and pETM6 for T7 induction) reaches low biomass levels in all three types of media. This makes sense because these conditions slow growth. In FIG. 22F, under the same conditions as panel E, only LB with IPTG induction was able to induce roGFP expression based on the excitation ratio compared to the empty vector panel. Based on the excitation ratio in the induced strain there is a wide range of cytoplasmic redox states (0.05-0.3), in addition to the range in biomass (1-15 OD600 nm). Therefore, even at conditions with low oxidative stress SHuffle has inconsistent growth, induction, and cytoplasmic oxidation.

Referring now to FIG. 23, E. coli with combined dynamic control of cytoplasmic reductases and DsbC overexpression (Agor; trxB-DAS4+; dsbC) does not have the same robustness challenges as SHuffle in micro-fermentations. FIG. 23A, the dynamic control strain was grown at 37° C. with high aeration (300 rpm shaking speed) in AB autoinduction media for low phosphate induction. Color indicates plasmid with EV-pSMART in light and yibDp-roGFP-pSMART in dark. FIG. 23B, the dynamic control strain was also grown at 30° C. with high aeration (300 rpm shaking speed) in AB autoinduction media with the yibDp-roGFP-pSMART shown in dark. The dynamic control redox strain most similar to SHuffle (Agor; trxB-DAS4+; dsbC) was used in AB autoinduction micro-fermentations at 37° C. with high aeration. Compared to the empty vector control, yibDp-roGFP in the dynamically controlled strain is consistently induced with oxidative cytoplasm. This strain also achieved high biomass levels with fluctuations between 18-300D 600 nm. For comparison, the dynamic control redox strain was used in micro-fermentations at 30° C. with high aeration. Although decreasing the temperature decreased biomass levels. The biomass levels consistently reached OD 600 nm and induced roGFP with oxidative cytoplasm. Cumulatively dynamically controlling redox state and dsbC cytoplasmic expression improves growth, expression, and cytoplasmic redox state robustness compared to SHuffle.

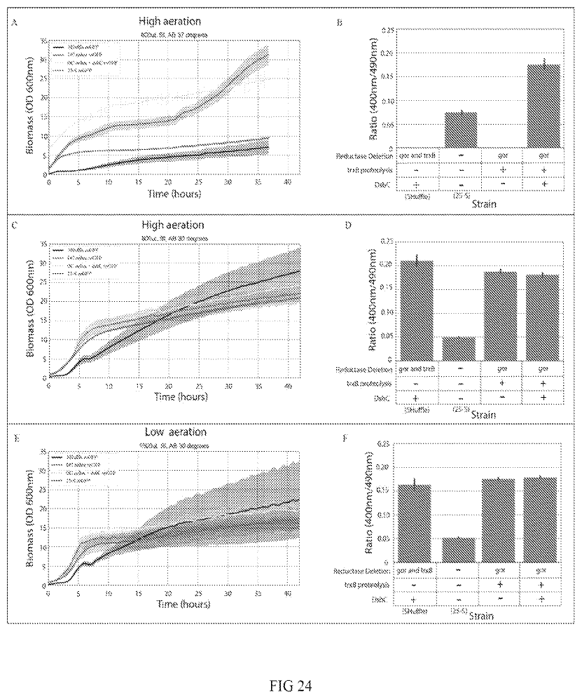

Referring to FIG. 24A-F, dynamic control of redox state improves strain robustness over reductase deletions in SHuffle E. coli based on biolector data is demonstrated. In FIG. 24A, Biolector growth curves of SHuffle and dynamic control strains (DC redox) with yibDp-roGFP-pSMART in AB autoinduction media Strains were incubated at 37° C. under high aeration conditions (800 μL fill volume). 25-S is the control strain with dynamic control capability but without reductase control. DC redox is the strain previously identified with the most oxidative cytoplasm (Agor; trxB-DAS4+) and DC redox+dsbC has dynamic overexpression of dsbC (Agor; trxB-DAS4+; dsbC). In FIG. 24B, the redox emission ratio was measured at the end of the biolector run from FIG. 24A with the corresponding strain modifications marked below each bar. Bars were omitted when roGFP did not induce. In FIG. 24C, Biolector growth curves are shown for these strains incubated at 30° C. under low aeration conditions (1500 μL fill volume). In FIG. 24D, the redox emission ratios were measured at the end of the biolector run from FIG. 24C. In FIG. 24E, Biolector growth curves for the strains incubated at 30° C. under low aeration conditions (1500 μL fill volume). In FIG. 24F, the endpoint redox emission ratio was measured at the end of the biolector experiment in panel E. Under high temperature and high aeration conditions, only the control strain and the dynamically controlled redox strain with dsbC can grow to high cell densities. Based on the redox ratio, the dynamically controlled strain has oxidative cytoplasm during these conditions. Therefore, the dynamically controlled strain has more robust growth and induction of roGFP, along with disulfide bond oxidation. Under low temperature and high aeration conditions all of the strains are able to grow to high cell densities, however the dynamically controlled strains have more tightly controlled growth whereas SHuffle has more variable biomass levels. Both the SHuffle and dynamically controlled strains induce roGFP expression with oxidative cytoplasm. Therefore, SHuffle requires more specialized growth conditions to express redox sensitive proteins. Under low temperature and low aeration conditions the strains grow to lower ODs. Again, the dynamically controlled strains have more tightly controlled growth whereas SHuffle has more variable biomass. Both the SHuffle and dynamically controlled strains induce roGFP expression with oxidative cytoplasm.

### Example 13: Expression of a Nanobody Employing Dynamic Control of Redox State E. coli Strain

Use of dynamic control of redox strains for expression of antibody fragments, a shake flask protocol adopted from Menancho-Melgar et al Biotechniques 2021 November; 71(5):566-572 was applied. Briefly, an overnight LB culture of the strain with the pSMART expression plasmid was used to inoculate 20 mL of AB autoinduction media in vented baffled 250 mL Erlenmeyer flasks (VWR, cat. no. 89095-270) and incubated at 37° C. and 150 r.p.m. for 24 hours. Flasks incubated at 30° C. and 150 r.p.m. were incubated for 48 hours after inoculation.

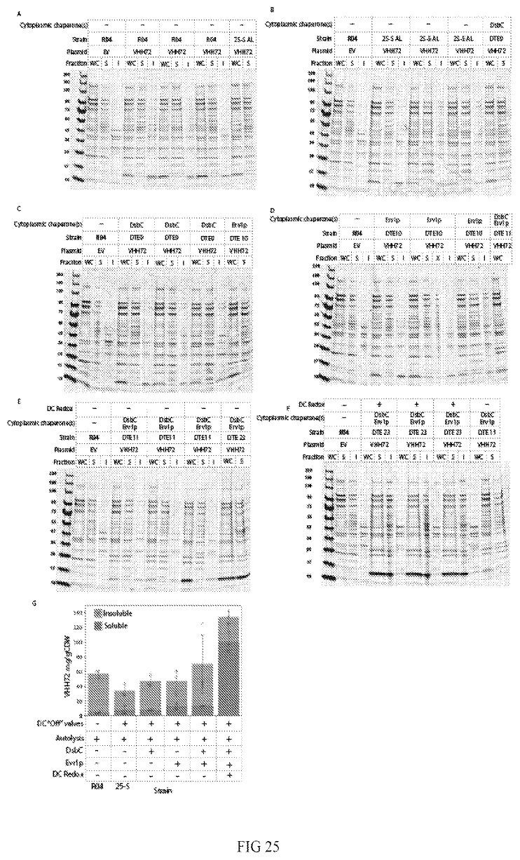

Referring to FIG. 25, shake-flask expression of the anti-SARS-CoV2 nanobody VHH72 (MW˜14 kDa) at 37° C. in E. coli strains with autolysis machinery, dynamic control of redox state, and chaperones DsbC and Evr1p is demonstrated. The impact of all of these individual components on VHH72 expression, as well as synergistic effects were analyzed. Specifically, the overall expression level in each strain was quantified based on the whole cell expression level. Subsequently this expression was stratified into soluble and insoluble fractions. Expression was performed in triplicate and fractions are labeled as whole cell (WC), soluble (S) and insoluble (I) In FIG. 25A, SDS-PAGE triplicate expression of the VHH72 nanobody in the autolysis strain (R04) without redox control. In FIG. 25B, SDS-PAGE triplicate expression of the VHH72 nanobody in the control strain (25-S AL) without redox control, but with DC “off” valve proteolysis and silencing capability as well as autolysis machinery. In FIG. 25C, SDS-PAGE triplicate expression of VHH72 in the 25-S AL strain modified with dynamic overexpression of dsbC (labeled DTE9). In FIG. 25D, SDS-PAGE triplicate expression of VHH72 in the 25-S AL strain modified with dynamic overexpression of Evr1p (labeled DTE10). In FIG. 25E, SDS-PAGE triplicate expression of VHH72 in the 25-S AL strain modified with dynamic overexpression of both dsbC and Evr1p (labeled DTE11). In FIG. 25F, SDS-PAGE triplicate expression of VHH72 in the 25-S AL strain modified with dynamic overexpression of both dsbC and Evr1p combined with dynamic control of redox state (labeled DTE23). In FIG. 25G, Summary of SDS-PAGE data from A-F quantifying overall VHH72 expression level in each strain, stratified into the soluble and insoluble fraction.

In conclusion, combining dynamic control of redox state with dynamic overexpression of both chaperones dsbC and Evr1p significantly increases the overall expression level of VHH72 in the strain compared to introducing overexpression of chaperones individually. Combining dynamic control of redox state with dynamic overexpression of both chaperones dsbC and Evr1p shifts VHH72 from predominantly insoluble expression to predominantly soluble expression.

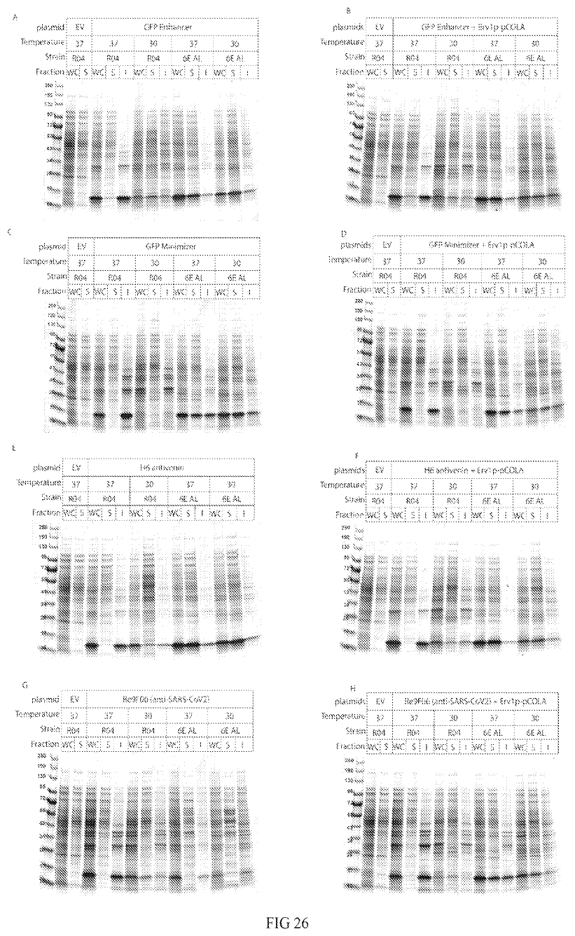

Referring now to FIG. 26, shake-flask expression of four additional nanobodies (MW˜14 kDa) in the autolysis strain (R04) without redox control, or in the 25-S modified strain that combines autolysis with dynamic redox control and overexpression of dsbC and Evr1p (labeled 6E AL) is demonstrated. Nanobody expression was compared in each strain with shake-flask expression performed at 37° C. and 30° C. Expression was separated into the whole cell fraction (WC) for overall expression level, soluble expression (S) and insoluble expression (I). Additionally increasing Evr1p expression level was performed in each strain with plasmid expression of Evr1p (Evr1p-pCOLA) to analyze the impact on nanobody expression and solubility. In FIG. 26A, SDS-PAGE expression of a GFP enhancer nanobody in strains R04 and 6EAL at both 37° C. and 30° C. In FIG. 26B, SDS-PAGE expression of a GFP enhancer nanobody in strains R04 and 6EAL with the addition of plasmid Evr1p (Evr1p-pCOLA) at both 37° C. and 30° C. In FIG. 26C, SDS-PAGE expression of a GFP minimizer nanobody in strains R04 and 6EAL at both 37° C. and 30° C. In FIG. 26D, SDS-PAGE expression of a GFP minimizer nanobody in strains R04 and 6EAL with the addition of plasmid Evr1p (Evr1p-pCOLA) at both 37° C. and 30° C. In FIG. 26E, SDS-PAGE expression of an antivenin nanobody (H6) in strains R04 and 6EAL at both 37° C. and 30° C. In FIG. 26F, SDS-PAGE expression of an antivenin nanobody (H6) with the addition of plasmid Evr1p (Evr1p-pCOLA) in strains R04 and 6EAL at both 37° C. and 30° C. In FIG. 26G, SDS-PAGE expression of an anti-Sars-CoV2 nanobody (Re9F06) in strains R04 and 6EAL at both 37° C. and 30° C. In FIG. 26H, SDS-PAGE expression of an anti-Sars-CoV2 nanobody (Re9F06) with the addition of plasmid Evr1p (Evr1p-pCOLA) in strains R04 and 6EAL at both 37° C. and 30° C. In conclusion, for the GFP enhancer, soluble expression was observed at 37° C. with 6EAL. There is some soluble with R04 but it is primarily insoluble at 37° C. Both strains have soluble expression with lower temperature. With respect to the GFP minimizer, soluble expression at 37° C. with 6EAL. With respect to H6 antivenin, soluble expression at 37° C. with 6EAL but there is some soluble expression in R04 at 30° C. With respect to Re9F06 (anti-SARS-Cov2, thermostable), soluble expression in R04 at 30° C. Adding plasmid Erv1p to 6EAL leads to soluble expression at 37° C. and 30° C.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method of expressing a heterologous protein product in a microorganism, the protein requiring at least one disulfide bond for proper folding of the protein, the method comprising:
(a) providing a genetically modified microorganism, the genetically modified microorganism comprising:
means for conditional expression or overexpression of a heterologous protein product;
a synthetic metabolic valve(s) comprising:
a gene expression-silencing synthetic metabolic valve characterized by silencing gene expression of one or more genes encoding one or more enzymes; or
an enzymatic degradation synthetic metabolic valve characterized by inducing enzymatic degradation of one or more enzymes, or
a combination thereof.
wherein the one or more enzymes of each synthetic metabolic valve(s) are the same or different; and
wherein reducing expression of at least one of the enzymes of a synthetic metabolic valve(s) is effective for altering the redox characteristics of the cytosolic environment of the genetically modified microorganism.
(b) growing the genetically modified microorganism in a growth media comprising a limiting nutrient,
wherein the microorganism maintains a reducing cytosolic environment during the growth phase comparable to the reducing cytosolic environment of a microorganism lacking genetic modifications;
(c) transitioning from a growth phase to a stationary and protein product producing phase, the transition comprising:
stopping the growth of the microorganism;
inducing the synthetic metabolic valve(s) resulting in a shift to an oxidative cytosolic environment;
inducing expression or overexpression of the heterologous protein product, thereby producing the protein product under oxidative conditions so that the necessary disulfide bonds are formed for proper folding of the heterologous protein product in the oxidative environment of the stationary phased genetically modified microorganism produced by induction of the synthetic metabolic valve(s).

2. The method of claim 1, wherein the genetically modified microorganism further comprises a chromosomal deletion of a gor, trxB, or gsh A gene.

3. The method of claim 1, wherein the synthetic metabolic valve(s) of the genetically modified microorganism are configured to reduce the activity of glutathione reductase, a thioredoxin reductase and a glutamate-cysteine ligase by transcriptional silencing of the glutathione reductase, thioredoxin reductase or glutamate-cysteine ligase gene, reducing the amount of glutathione reductase, thioredoxin reductase or glutamate-cysteine ligase enzyme by selective proteolysis, or both.

4. The method of claim 1, wherein the synthetic metabolic valve(s) of the genetically modified microorganism are configured to regulate gor, trxB and gshA by transcriptional silencing of the gor, trxB or gshA gene, reducing the amount of gor, trxB or gshA enzyme by selective proteolysis, or combinations thereof.

5. The method of claim 4, wherein a synthetic metabolic valve that, when induced causes the selective degradation of trxB, the synthetic metabolic valve comprising a phoB promotor.

6. The method of claim 1, wherein the genetically modified microorganism is configured to conditionally overexpress a thiol:disulfide interchange protein, a thiol oxidase, or a combination thereof in the stationary phase.

7. The method of claim 1, wherein the genetically modified microorganism is configured to conditionally overexpress a dsbC gene, a evr1 gene, or a combination thereof in the stationary phase.

8. The method of claim 1, wherein the heterologous protein product is a human protein, an antibody, an antibody fragment, a single chain variable fragment of an antibody, a nanobody, a protein that is approximately 14 kDa in size, a hyaluronidase-I, or a tissue plasminogen activator.

9. The method of claim 1, wherein microorganism growth is stopped in the transition phrase by phosphate depletion of the growth media.

10. The method of claim 1, wherein the genetically modified microorganism is an E. coli microorganism.

11. The method of claim 1, wherein the silencing synthetic metabolic valve silences a gene encoding an enzyme selected from the group: enoyl-ACP reductase (fabI), citrate synthase (gitA), soluble transhydrogenase (udhA), glucose-6-phosphate-l-dehydrogenase (zwf), or lipoamide dehydrogenase (lpd), or combinations thereof;
or the enzyme of the enzymatic degradation synthetic metabolic valve is selected from the group of: enoyl-ACP reductase (fabI), citrate synthase (gitA), soluble transhydrogenase (udhA), glucose-6-phosphate-l-dehydrogenase (zwf), or lipoamide dehydrogenase (lpd), or combinations thereof;

12. The method of claim 11, wherein the silencing synthetic metabolic valve encodes a soluble transhydrogenase (udhA) gene operatively linked to a yibD, ugpB, ydfH, phoB, or pstS promotor.

13. The method of claim 1, wherein the synthetic metabolic valve comprises:
(i) a gene encoding at least one small guide RNA specific for targeting more than one gene;
(ii) a Cascade protein complex wherein cas3 is absent or cas3 is modified to form part of the Cascade protein complex but also lacks enzyme activity,
wherein the (i) gene and the (ii) Cascade protein complex are inhibited or suppressed when inorganic phosphate is available to the microorganism, but are activated in the absence of inorganic phosphate.

14. A genetically modified microorganism comprising:
means for conditional expression or overexpression of a heterologous protein product;
a synthetic metabolic valve(s) comprising:
a gene expression-silencing synthetic metabolic valve characterized by silencing gene expression of one or more genes encoding one or more enzymes; or
an enzymatic degradation synthetic metabolic valve characterized by inducing enzymatic degradation of one or more enzymes, or
a combination thereof.
wherein the one or more enzymes of each synthetic metabolic valve(s) are the same or different; and
wherein reducing expression of at least one of the enzymes of a synthetic metabolic valve(s) is effective for altering the redox characteristics of the cytosolic environment of the genetically modified microorganism,
wherein the microorganism grows in a media while maintaining a reducing cytosolic environment during the growth phase, the reducing cytosolic environment comparable to the reducing cytosolic environment of a microorganism lacking genetic modifications; and
wherein the synthetic metabolic valve(s) of the genetically modified microorganism, when activated, produce an oxidative cytosolic environment to promote formation of disulfide bonds for proper folding of the heterologous protein product.

15. The genetically modified microorganism of claim 14, wherein the genetically modified microorganism further comprises a chromosomal deletion of a gor, trxB, or gsh Agene.

16. The genetically modified microorganism of claim 14, wherein the synthetic metabolic valve(s) of the genetically modified microorganism are configured to reduce the activity of a thioredoxin reductase and a glutamate-cysteine ligase by transcriptional silencing of the thioredoxin reductase or glutamate-cysteine ligase gene, reducing the amount of thioredoxin reductase or glutamate-cysteine ligase enzyme by selective proteolysis, or both.

17. The genetically modified microorganism of claim 14, wherein the synthetic metabolic valve(s) of the genetically modified microorganism are configured to regulate trxB and gshA by transcriptional silencing of the trxB or gshA gene, reducing the amount of trxB or gshA enzyme by selective proteolysis, or combinations thereof.

18. The genetically modified microorganism of claim 14, wherein the genetically modified microorganism is configured to conditionally overexpress a thiol:disulfide interchange protein, a thiol oxidase, or a combination thereof in the stationary phase.

19. The genetically modified microorganism of claim 14, wherein the genetically modified microorganism is configured to conditionally overexpress a dsbC gene, a evr1 gene, or a combination thereof in the stationary phase.

20. The genetically modified microorganism of claim 14, wherein the heterologous protein product is a human protein, an antibody, an antibody fragment, a single chain variable fragment of an antibody, a hyaluronidase-I, or a tissue plasminogen activator.

21. The genetically modified microorganism of claim 14, wherein microorganism growth is stopped in the transition phrase by phosphate depletion of the growth media.

22. The genetically modified microorganism of claim 14, wherein the genetically modified microorganism is an E. coli microorganism.

23. The genetically modified microorganism of claim 14, wherein the gene of the silencing synthetic metabolic valve encodes an enzyme selected from the group: enoyl-ACP reductase (fabI), citrate synthase (gitA), soluble transhydrogenase (udhA), glucose-6-phosphate-l-dehydrogenase (zwf), or lipoamide dehydrogenase (lpd), or combinations thereof;
or the enzyme of the enzymatic degradation synthetic metabolic valve is selected from the group of: enoyl-ACP reductase (fabI), citrate synthase (gitA), soluble transhydrogenase (udhA), glucose-6-phosphate-l-dehydrogenase (zwf), or lipoamide dehydrogenase (lpd), or combinations thereof;

24. The genetically modified microorganism of claim 14, wherein the synthetic metabolic valve comprises:
(i) a gene encoding at least one small guide RNA specific for targeting more than one gene;
(ii) a Cascade protein complex wherein cas3 is absent or cas3 is modified to form part of the Cascade protein complex but also lacks enzyme activity,
wherein the (i) gene and the (ii) Cascade protein complex are inhibited or suppressed when inorganic phosphate is available to the microorganism, but are activated in the absence of inorganic phosphate.
